(19) United States
(12) Patent Application Publication
(10) Pub. No.: US 2007/0213661 A1
(43) Pub. Date: Sep. 13, 2007

(54) Closure system for managing rectal or anal incontinence

(76) Inventor: Fred Gobel, Wilhelmsfeld (DE)

Correspondence Address: Pauley Petersen & Erickson, Suite 365, 2800 W. Higgins Road, Hoffman Estates, IL, 60195, US

(21) Appl. No.: 11/373,830

(22) Filed: Mar. 10, 2006

(51) Int. Cl. A61M 29/00

(52) U.S. Cl. 604/96.01

(57) ABSTRACT

A closure system for managing rectal or anal incontinence, having a shaft element, with a distal end and a proximal end and at least one lumen. An inflatable intrarectal balloon includes a flexible, extensible plastic material with a distal end and a proximal end, each of which is attached to the shaft element in a securely bonded and sealed fashion. The intrarectal balloon communicates with the at least one lumen via a perforation in the wall of the shaft element and can be acted on with a filling medium via the lumen. An anchoring element serves as a buttress, which is attached to the shaft element spaced apart from the intrarectal balloon on its proximal side, in the direction toward the proximal end of the shaft element. The distal end of the intrarectal balloon is attached to the shaft element, the balloon being invaginated in the direction toward the proximal end of the balloon in such a way that the intrarectal balloon attached with its two ends to the shaft element, as it is filled with a filling medium, executes a rolling motion from the distal end of the shaft element toward the proximal end of the shaft element.

### BACKGROUND OF THE INVENTION

1. Field of the Invention

This invention relates to a closure system for managing rectal or anal incontinence, having a shaft with a distal end and a proximal end and at least one lumen, an inflatable intrarectal balloon with a flexible, extensible plastic material with a distal end and a proximal end, each of which is attached to the shaft element in a securely bonded, sealed fashion. The intrarectal balloon communicates with the at least one lumen via a perforation in the wall of the shaft element and can be acted on with a filling medium via the lumen. An anchoring element serving as a buttressing element is attached to the shaft element spaced apart from the intrarectal balloon on its proximal side, in the direction toward the proximal end of the shaft element.

2. Discussion of Related Art

Caring for patients with rectal or anal incontinence using suitable closure or tampon systems in a manner that permits an undisturbed daily routine has been unsatisfactory up to this point despite many proposed designs. The large majority of those affected are thus still instructed to use diapers or special incontinence systems.

Continence-producing rectal closure systems have up to now essentially been brought to market in two basic types.

The most frequently used are rectal tampons made of expanding, automatically generating foam or cellulose material. These are inserted by the patient through the anus into the rectum in the form of compressed, approximately suppository-shaped bodies coated with a lubricant and expand there as they absorb digestive secretions, sometimes growing to several times their original volume. In order to improve the sealing action, expanding tampons are often supplied in the form of a half shell, with the convexity oriented toward the anus and fitting snugly into the terminal section of the rectum (rectum ampulla). The concavity points in a bowl shape toward the rectosigmoid segment of the intestine.

One disadvantage during use of such expanding bodies is that in many cases, the removal of the swollen tampon body is difficult and often painful for the patient. As a rule, the patient removes the tampon by a pull line extending through the anus. Injuries to the sensitive anal canal by the expanding body are not unusual. The bulging tampon body that has expanded in the rectum can also be irritating to the patient and can cause an urge to empty bowels and trigger the defecation reflex. Expanding tampons are a non-cleanable, non-reusable single-use article. Since the rectal tampons are often changed several times a day, the consumption of tampons can be considerable (up to 10 per day).

In addition to expanding bodies, there are also known tamponades based on rectally placed, externally inflatable balloon bodies. As a rule, the balloons produce a seal in the terminal segment of the rectum through direct, all-over contact with the wall of the organ. Corresponding balloon tamponades have been disclosed, for example, produced in the form of simple balloon catheters. The balloon body is expanded in the rectum, taking up space therein, and is pulled toward the anus into the terminal ampulla by the catheter shaft extending through the anus. A suitable stopper or anchoring mechanism outside or in front of the anus can fix and hold the balloon body in its position at the bottom of the rectum ampulla.

As a rule, conventional rectally sealing balloon bodies are inflated with a relatively high filling pressure and sometimes exert considerable stress on the tissue of the rectum. In many cases, the organ is spherically deformed and expanded. The tissue circulation can fall victim to the forces exerted, resulting in ulcers and lasting, troublesome structural and functional lesions on the organ. The filled bodies are also perceived as foreign bodies by the patient and can be accompanied by a permanent or briefly intermittent urge to empty one's bowels. The use intervals of such balloon tamponades are therefore limited as a rule; continuous, long-lasting use cannot be recommended due to the not insignificant risk of trauma.

German Patent Reference DE 102 55 065 A1 discloses a balloon catheter for transurethral insertion into the bladder, having an elastic catheter shaft with an inflatable balloon element attached to it, which is connected to an inflation duct incorporated into the wall of the catheter shaft, for conveying urine out of the bladder.

Species-defining closure systems for managing rectal or anal incontinence are disclosed by Japanese Patent Reference JP 10-234854 A, Japanese Patent Reference JP 09-253112 A, and German Patent Reference DE 44 36 796 C2. These known closure systems use an intrarectal inflatable balloon as a closure device that expands only radially when inflated so that the above-mentioned disadvantages and problems for the patient also occur in these embodiment forms.

### SUMMARY OF THE INVENTION

One object of this invention is to provide a space-occupying seal that encompasses the entire terminal rectum with the aid of a closure system, which largely avoids the generation of forces exerted against and directed toward the lateral portions of the rectum ampulla by conventional balloon seals and largely avoids a risk of trauma in long-term use.

This object is attained according to this invention with a closure system for managing rectal and anal incontinence having characteristics taught in this specification and in the claims.

The closure system for managing rectal and anal incontinence according to this invention is distinguished by the fact that during invagination of the balloon in the direction of the proximal end of the balloon, the distal end of the intrarectal balloon is attached to the shaft element so that as it is being filled with a filling medium, the intrarectal balloon, which is fastened to the shaft element at both of its ends, executes a rolling motion from the distal end of the shaft element in the direction of the proximal end of the shaft element.

Modifications of the closure system according to this invention are discussed in this specification and in the dependent claims.

By contrast with the prior balloon-based tampon systems that produce a space-occupying seal encompassing the entire terminal rectum, the closure according to this invention produces a seal by a closure mechanism that acts on the anal canal or on the tissue surrounding the anal canal (anal ring). The diffuse generation of forces exerted against and directed radially toward the lateral portions of the rectum ampulla by conventional balloon seals is largely avoided because a focused seal is produced, which is predominantly directed in the axial direction from the inside, toward the base of the rectum or the anal ring.

The axially acting sealing mechanism according to this invention is achieved by a particular shape and placement of the rectally sealing intrarectal balloon on the shaft element that supports the balloon. The function of the anal sealing mechanism is explained.

The closure system according to this invention comprises three main components.

One component is formed by the shaft element, which passes through the anal canal and supports the intrarectally sealing balloon. It is preferably manufactured of a somatically tolerable plastic that is as soft as possible but which must assure sufficient stability to overcome the resistance of the anus during insertion of the sealing balloon without bending of the shaft. Its front end is atraumatically rounded. The shaft body can, for example, be comprised of a segment of soft PVC tube.

The intrarectally sealing balloon body is attached to the front end of the shaft piece and comprises a somatically tolerable soft film with a shape and dimensions that correspond fully or almost fully to those of the final balloon dimensions. Preferably, the balloon is made of a film that is as thin as possible. Films of this kind, for example, include polyurethane, and can be manufactured in a wall thickness range of a few micrometers using the so-called blow-molding method. The balloon material ideally has a low compliance that is significantly lower than that of materials similar to silicon or latex. Even at a high inflation pressure, the balloon film should not expand out into a spherical shape as is typical for latex and silicon, but should instead maintain the preferred cylindrical shape. Polyurethane-based balloon films in the microscopically thin wall thickness range have a characteristic that corresponds to the function of the closure.

Outside the body, directly in front of the anus or lying in the anal cleft, an anchor element serving as a buttressing element is attached to the shaft piece and has a variable shape. The anchor element comprises, for example, a disk-shaped or spherical balloon body that is likewise made of soft film material and rests against the anus in a gentle, non-irritating fashion. The intrarectally sealing balloon and the balloon serving as a buttressing element placed in front of the anus can be inflated either separately or by a shared inflation duct and/or can communicate with each other via this duct.

The body serving as a buttressing element and placed in front of the anus can alternatively be embodied as a support piece that is attached in a T configuration at right angles to the shaft piece and that comes to rest against the anal cleft. It can also conceivably be embodied in the form of a ball-shaped or conical element made of foam or also as a wedge made of soft PVC, pressed cellulose, or foam that rests against the anal cleft.

For the focused balloon seal according to this invention, which acts on the anus in the axial direction and largely avoids a radial expansion, the intrarectal balloon body is preferably embodied in a cylindrical form with a diameter of approx. 3 to 6 cm and a length of approx. 5 to 10 cm.

The lower balloon the end of the intrarectal balloon is affixed to the shaft element at a point spaced approximately 2 to 4 cm apart from the anchor element or the buttressing body. The upper balloon end oriented toward the tip of the shaft body is shifted with respect to the fixed lower end of the shaft element, to such a degree that the resulting distance corresponds to 70% to 50% of the cylinder length of the inflated, but unmounted balloon body in the rest position. For the function of the closure system according to this invention, it is advantageous if a fixing point of the upper balloon end is shifted as far as possible toward the lower balloon end on the shaft element.

The use of a cylindrical or almost cylindrically shaped intrarectal balloon body and the above-described specific type of shifting or invagination of the distal balloon end, provides the balloon with a tendency, as it inflates, to position itself on the shaft element so that its two balloon ends move to the center of the balloon being generated until the inflated balloon lies against the shaft in its lowest stress state. This results in a rolling motion of the balloon oriented away from the distal end of the shaft element relative to the immobilizing anchor element at the other end.

As a result, with an axially oriented opposite motion and axially acting pressure, the balloon nestles snugly against the anal ring from the inside in an ideally sealing, minimally irritating fashion. The pressure against the anus can be conveniently controlled by the patient during the filling of the balloon and adjusted according to individual needs.

The thinner the walls of the balloon film are, the less force is exerted on the anal ring. When micro-thin balloon films of polyurethane are used, such as of the material types Pellethane® 2363 from Dow Chemical or Estane®, with a wall thickness of approximately 0.005 to 0.015 mm, balloon inflation pressures of approx. 10 to 20 mbar are enough to achieve a sufficient rectal seal.

With such low filling pressures, even with very long periods of use, there is no need to fear a diminution of the mucous membrane perfusion whose capillary circulatory pressure lies between 25 and 35 mbar as a rule. Typical inflation pressure associated lesions or irritations caused by conventional balloon closures can thus be avoided.

Due to the considerable limitation of the pressure on the anus, the sealing body, which does not primarily take up space and is acted on with a low pressure, as a rule does not trigger a desire to defecate and is not perceived as irritating by the patient.

If the shaft element has an open distal end and a lumen that extends from the proximal end to the distal end, then it is suitable for use, for example as an inlet tube, with the intrarectal balloon simultaneously assuring a good sealing of the rectum.

The adjustable filling pressure of the balloon makes the closure system according to this invention also suitable for temporary treatment of hemorrhoidal hemorrhages because the inflation pressure of the balloon on the one hand has a hemorrhage-arresting action and on the other hand, prevents the intestinal content from coming into contact with the surface of the wound, thus preventing infection.

The closure system according to this invention is also suitable for protecting against anastomoses in corresponding surgical procedures.

Finally, the closure system according to this invention, due to the rolling motion of the intrarectal balloon in the direction toward the proximal end as it inflates, is also suitable for spreading open the anus and temporarily pushing back the tissue in the region of the rectum of a patient, for example during radiation therapy.

### BRIEF DESCRIPTION OF THE DRAWINGS

Exemplary embodiments of this invention are explained in view of the drawings, wherein:

FIG. 1 shows a longitudinal section taken through a closure system for rectal closure;

FIG. 2 shows an intrarectal balloon in a loosely inflated rest state;

FIG. 3 shows a shaft element with the intrarectal balloon attached to it and being withdrawn;

FIG. 4a shows the rectal closure according to FIG. 1 in the rest state, placed in a terminal rectum;

FIG. 4b shows the rectal closure according to FIG. 4a, but in a pressurized state;

FIG. 5 shows a closure system in the terminal rectum in the pressurized state, with a shaft element having an inserted probe;

FIG. 6a shows a closure system with an integrally designed balloon body in the terminal rectum, in the rest state and in the pressurized state;

FIG. 6b shows a schematic, transverse section S taken through the anal canal, showing a design of reserve folds of the balloon body according to FIG. 6a;

FIG. 7 shows the anchor element according to FIG. 1, with the shaft element;

FIGS. 8a to 8d show various embodiments of an anchor element for the closure system;

FIG. 9a shows a disk-shaped anchoring element for the closure system in the rest state; and

FIG. 9b shows the closure system with the anchor element according to FIG. 9a, when in use, in a stressed state.

### DESCRIPTION OF PREFERRED EMBODIMENTS

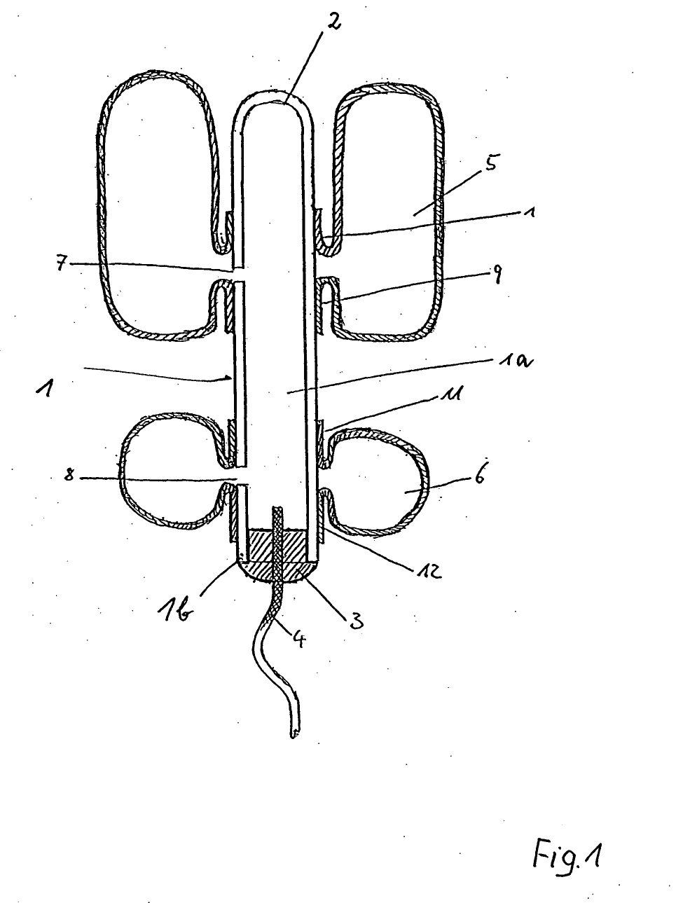

FIG. 1 schematically shows one preferred embodiment of the closure system according to this invention, in the form of a rectal closure for managing rectal and anal incontinence. The shaft element 1 is embodied in a form of a tube with a circular cross-section, having one end oriented toward the rectum, for example its distal end, and closed by an atraumatically rounded tip 2. Ideally, the shaft element 1 should not exceed an outer diameter of approx. 12 mm. The shaft element lumen 1a extending from the atraumatic distal end 2 of the shaft element to the proximal end 1b is closed, for example by a cap, at the open proximal end 1b, which lies outside the body during use. The inflation line 4 is routed through a sealing body 3 and can fill the lumen 1b and the balloon with which it communicates with filling medium. The end of the inflation line 4 has a closure valve, not shown here, suitable for use of gas and fluid as the filling medium. The valve is integrated into a so-called pilot balloon that makes it possible for the patient to estimate by touch the respective inflation state or inflation pressure of the balloon when it is inside or outside the body. The intrarectal balloon 5 that is situated close to the distal end 2 of the shaft element 1 and a buttressing balloon 6 serving as an anchoring element that is situated close to the proximal end 1b of the shaft element 1 are attached to the shaft element 1 in a sealed fashion, spaced apart from each other. The intrarectal balloon 5 is connected to the lumen 1a via a perforation 7 in a wall of the shaft element 1 and the buttressing balloon 6 is connected to the lumen 1a via the perforation 8 in the wall of the shaft element 1 and the balloons 5, 6 are inflated at the same time via the inflation line 4 and the lumen 1a. The distance between the perforations 7 and 8 of the intrarectal balloon 5 and the buttressing balloon 6 ideally lies between approx. 2 and 4 cm. It essentially corresponds to the length of the anal canal of a patient. In order to basically correspond to the respective dimensions of the anal canal of various patients, this invention proposes manufacturing and using for example two embodiments of the rectal closure according to FIG. 1, one with a distance of 2 cm between the two perforations and one with a distance of 4 cm between the two perforations of the shaft element 1.

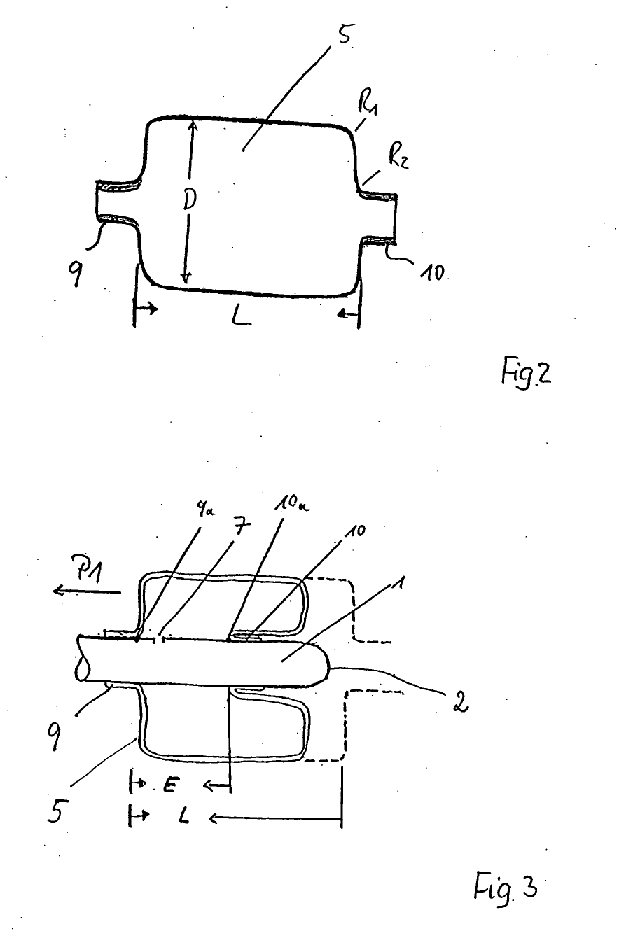

In FIG. 2, the intrarectal balloon 5 of the closure system according to FIG. 1 is depicted as a loosely inflated component in the rest state, when not mounted on the shaft element. The intrarectal balloon 5 comprises an extruded raw tube segment with a preferable wall thickness in a range from 80 to 140 μm, made of a flexible, extensible plastic material, in particular a polyurethane material, and blown into an approximate cylinder or roller shape using the so-called blow molding method. The wall thickness of the balloon here is reduced to 5 to 25 μm, preferably 5 to 15 μm, while the remaining tube ends 9, 10 at the ends of the cylinder are expanded by only approximately half the wall thickness of the raw tube segment, to a wall thickness of approx. 40 to 60 μm and are thus correspondingly thicker. The inner diameter of the tube ends is preferably slightly smaller than the outer diameter of the shaft element 1. The balloon 5, with its two tube ends 9, 10 is attached to the shaft element 1, preferably by stretching the tube ends 9, 10 so that it rests against the shaft element 1 under pressure and is attached there permanently, for example with an additional adhesive or weld. It should also be noted that in the various figures, the tube ends of the balloon are not depicted as thicker, but they still have a significantly thicker wall thickness than the blown part of the balloon. The intrarectal balloon 5 is preferably embodied in the form of an approximate cylinder and has a length L determined by the two transition points of the balloon radii at the two balloon shoulders R1 and R2. The diameter in the cylindrical segment of the intrarectal balloon 5 is labeled D.

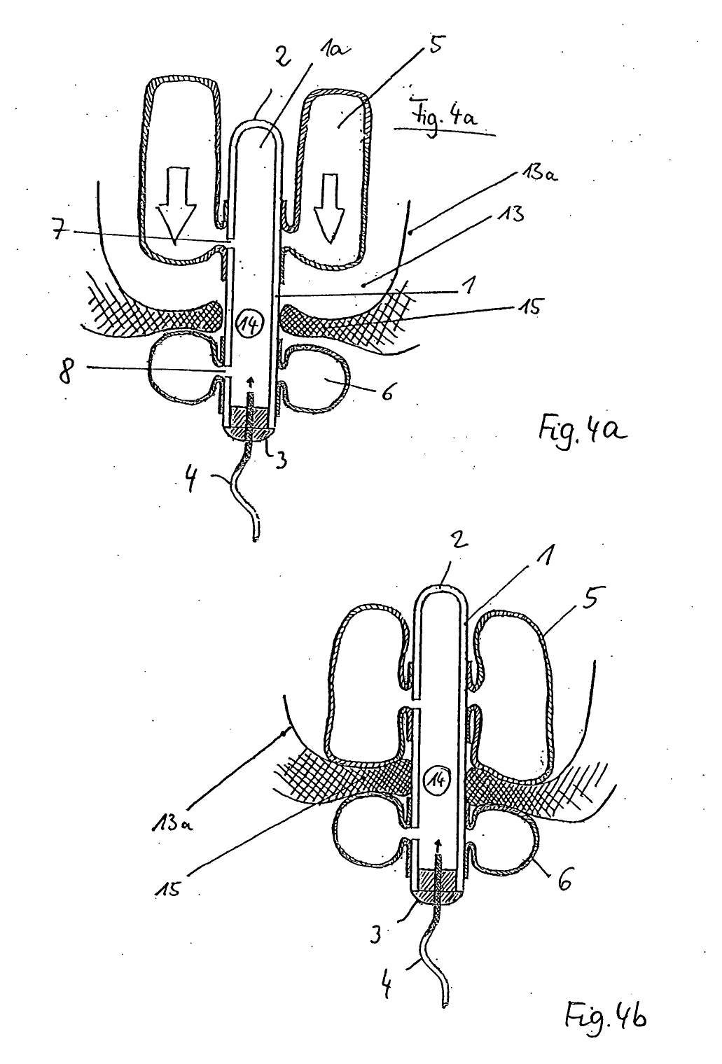

FIG. 3 shows the principal arrangement and attachment of the intrarectal balloon 5, according to FIG. 2, to the shaft element 1. In order to achieve a sufficient countermovement of the intrarectal balloon 5 that produces the axial sealing effect, such as a movement in the direction of the longitudinal axis of the shaft element 1, the distal attachment region of the distal tube end 10 of the intrarectal balloon 5 to the shaft element 1 is determined and embodied in the following manner. The distance E between the attachment point 9a of the proximal tube end 9 of the intrarectal balloon 5 and the attachment point 10a of the distal tube end 10 of the intrarectal balloon 5, respectively defined as a transition point of the balloon from the fixed tube end to the free, unfixed shaft shoulder R2, should be at most 70% of the length L of the loosely inflated, unmounted balloon 5 in the rest state, as described in connection with FIG. 2 and also depicted with dashed lines in FIG. 3. The function of the axially directed balloon countermovement of the rectal closure according to this invention is, however, most efficient if the distance between the perforation 7 in the shaft element, the inflation opening for the intrarectal balloon 5, and the attachment point 10a is less than 50% of the length L, down to 20% or less.

In order to achieve the greatest possible sliding distance or countermovement of the intrarectal balloon 5 in the arrow direction P1 toward the anal ring, in a particular embodiment of this invention, the proximal tube end 9 is attached in the immediate vicinity of the perforation 7, the inflation opening for the intrarectal balloon 5. The opposite, distal tube end 10 is shifted on the shaft element 1 toward the proximal tube end 9 of the intrarectal balloon 5, for example is invaginated into the balloon, to such an extent that it comes as close as possible to the perforation 7 and the perforation 7 is embodied in the shaft element 1, as close as possible to the proximal end of the balloon 5.

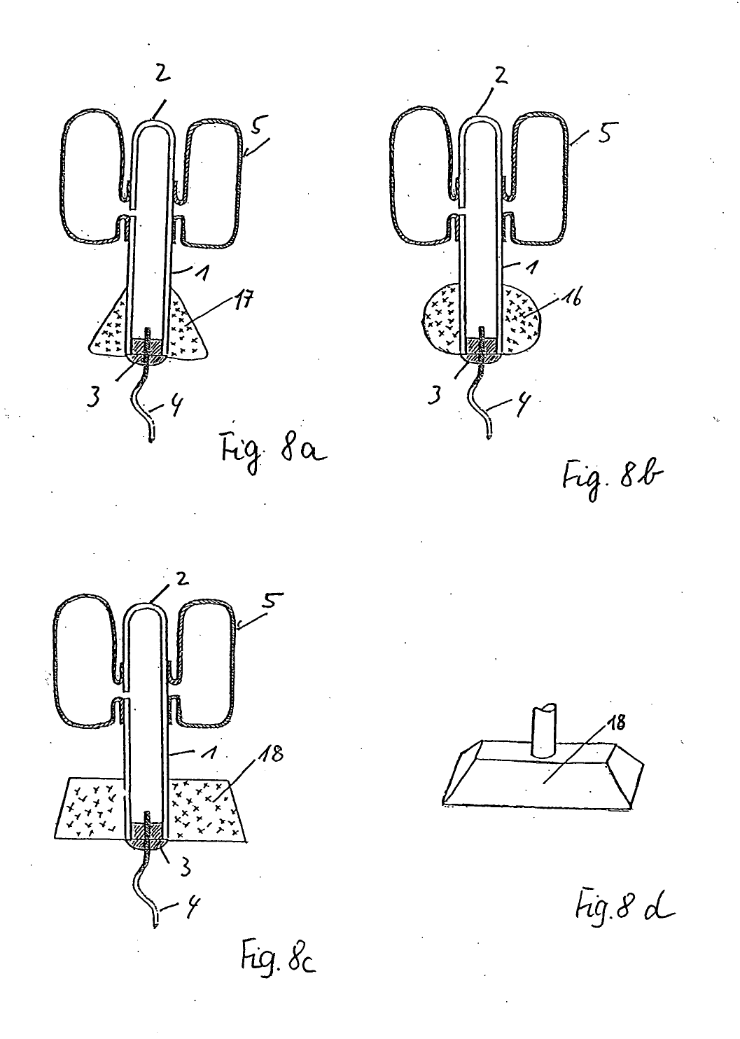

FIG. 7 also shows one preferred form of the anchoring element in its embodiment as a buttressing balloon 6 according to FIG. 1. When in the loosely inflated rest state, the buttressing balloon 6 is in the form of a flat, disk-shaped body. The buttressing balloon 6 is also made of an extruded tube segment comprising a flexible, extensible plastic material, preferably polyurethane, by blow molding, starting with a tube segment with a wall thickness of approx. 80 to 200 μm. The balloon is blown to a wall thickness of 5 to 45 μm, preferably 5 to 25 μm, leaving the tube ends at the ends of the disk-shaped body, which are blown to a lesser degree, producing a wall thickness of approx. 40 to 80 μm, for attachment to the shaft element with a correspondingly greater wall thickness. The buttressing balloon 6 is then pulled onto the shaft element 1 under pressure by stretching the tube ends 11, 12 and is attached to the shaft element 1 in a stationary fashion, for example by an additional adhesive or weld. The two tube ends 11, 12 of the buttressing balloon 6 are attached symmetrically to each other and close to the perforation 8 in the shaft element 1 used to inflate the buttressing balloon 6. Preferably, the two tube ends 11, 12 are attached the same distance from the transition points 11a, 12a of the balloon on the shaft element 1, close to the perforation 8, the inflation opening. The buttressing balloon 6 remains stationary and stabilizes in the radial direction when inflated with a filling medium. The above-described embodiment can be in the form of a micro-thin balloon body made of a soft film which is optimally tolerated in the anal region of the patient and is sufficient in its buttressing function for situations that come up in day-to-day life.

In lieu of inflating the intrarectal balloon 5 and the buttressing balloon 6 according to FIG. 1 via the central lumen 1a of the shaft element 1, it is also possible to inflate the two balloons by separate inflation ducts, which are integrated into the wall of the shaft element 1, for example, and thus to adjust the inflation pressure of the two balloons individually.

Alternatively, according to another embodiment of this invention, it is also possible to produce the two balloons 5 and 6 in an integrated form from a single contiguous tube segment using the blow molding method. In this case, the intrarectal balloon and the buttressing balloon are produced from an extruded tube segment with a wall thickness of 80 to 140 μm made of a flexible, extensible plastic material using the blow molding method. The balloons have a wall thickness of 5 to 45 μm and the tube ends at either end are blown to a lesser degree, to a wall thickness of approximately 20 to 80 μm. The two outer tube ends are used for stationary, sealed attachment to the shaft element. Between the two blown balloon bodies of the intrarectal balloon and the buttressing balloon, a non-blown tubular connecting piece remains, having a wall thickness for a spacing between the two balloon formations that corresponds to the wall thickness of the tube ends and is likewise pulled onto the shaft element and serves to fasten this section onto the shaft element in a stationary, sealing fashion. In the balloon produced in such an integrated fashion, including both the intrarectal balloon and the buttressing balloon spaced apart from it, the diameter of the intermediate connecting piece situated between the two balloons should be dimensioned so that it can be mounted onto the shaft element 1 under a powerful tension that prevents it from being dislocated, as a result of which, the balloons cannot reciprocally influence each other in their position on the shaft element and cannot move on the shaft element.

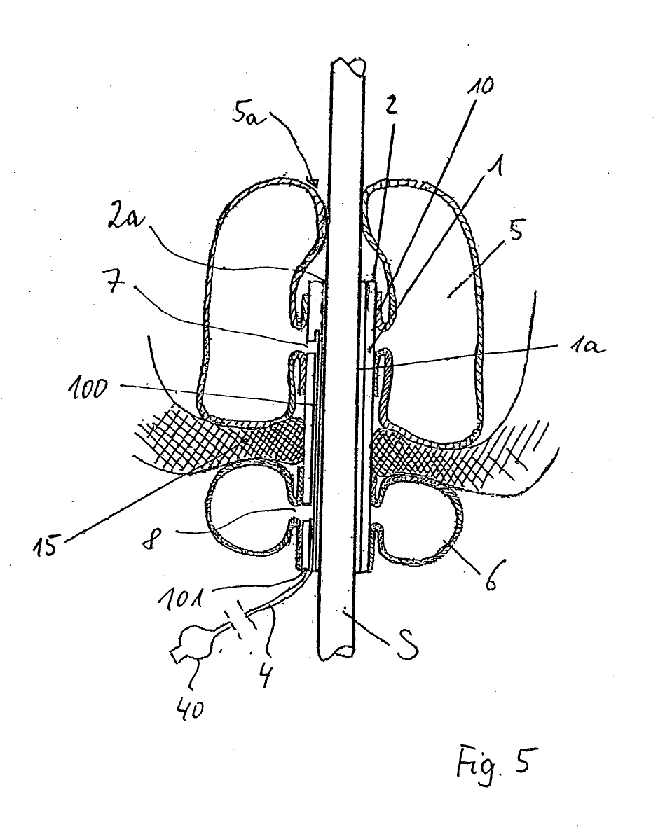

FIG. 4a schematically depicts the placement of the rectal closure according to FIG. 1 in the terminal rectum in the as yet unpressurized state, and FIG. 4b shows the pressurized state. The rectum ampulla 13 accommodates the intrarectal balloon 5. As the intrarectal balloon 5 and the buttressing balloon 6 are filled via the inflation line 14 by the lumen la of the shaft element 1 and the perforations 7 and 8 that connect the shaft lumen la to the balloons, the intrarectal balloon 5 moves in a quasi-rolling fashion away from the distal end, as shown by the arrows, toward the anus 14 and with its proximal end oriented toward the anus, invaginates over the anal ring 15 in a sealed fashion, such as shown in FIG. 4b. The force acts on the anus predominantly in the longitudinal direction of the shaft element 1, the axial direction, and the lateral portions of the rectum ampulla 13a remain largely untouched and are not subjected to any relevant expansion and pressure by the intrarectal balloon 5.

In the event of a fecal column pressing sigmoidally against the intrarectally placed balloon, the force presses against the intrarectal balloon 5 and causes a radial deformation of the balloon 5, with respect to the longitudinal axis of the shaft element, which increases the sealing action. The originally cylindrical balloon 5, such as shown in FIG. 4a, approaches the form of a spherical discoid that then also reaches the wall of the rectum and there produces a seal. As a rule, this deformation gives the patient the time necessary to defecate in a sanitary fashion.

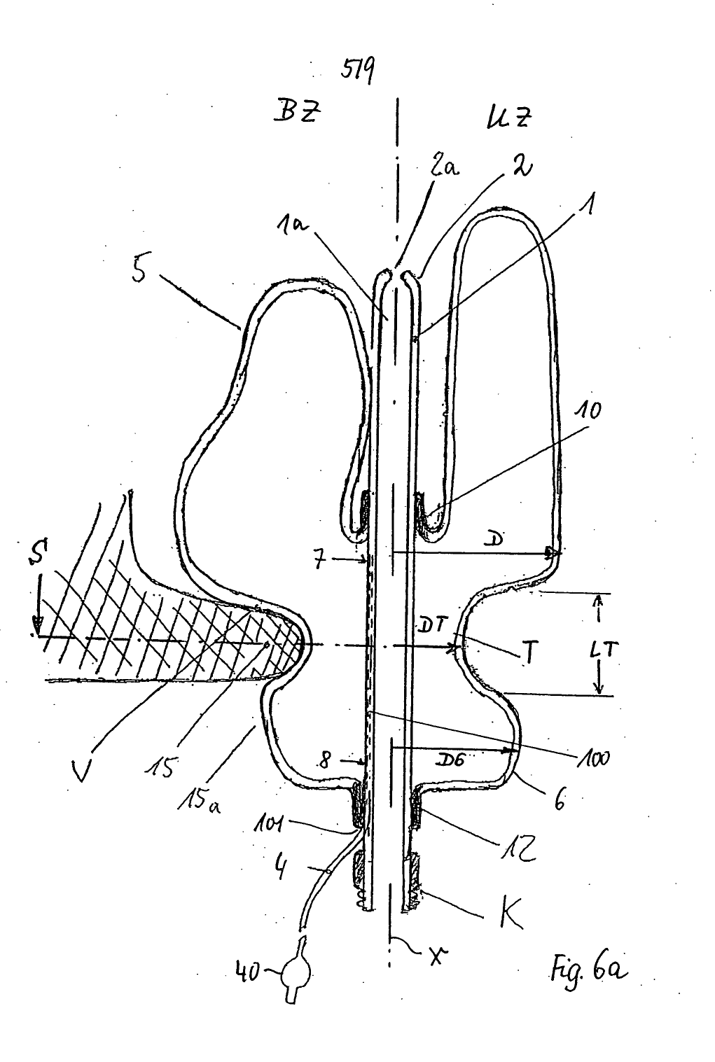

FIG. 5 shows a variant of the closure system in which the distal end 2 of the shaft element 1 has the opening 2a that permits the passage of a probe S through the shaft element 1 and the opening 2a is sealed to prevent the escape of gas and secretions from the digestive tract. The shaft element 1 at the distal end 2 is shortened so that it terminates at approximately the same height as the distal attachment region 10 of the intrarectal balloon 5. The balloon body that expands during inflation of the balloon 5 invaginates in circular fashion over the distal end 2 and forms a seal around the probe shaft S passing through it, such as shown in FIG. 5 as element 5a. Once a seal is produced, the probe shaft S can then be comfortably moved into and out of the intestine, without any relevant resistance due to the presence of the shaft element 1 in the intestine. This embodiment of the closure system according to this invention would be suitable, for example, for introducing endoscopes, which should move or slide with little resistance through the rectal closure, which is then functioning as an inductor or insertion point.

In a similar manner, the shaft element 1 in this embodiment can also be used as an inlet tube for introducing fluids into the intestine.

If the probe is removed from the shaft element 1, the distal end of the filled membrane-like balloon 5 closes, functioning as a valve, so to speak, thus preventing reflux of intestinal contents. With a corresponding dimensioning and embodiment, according to this invention, of the attachment of the intrarectal balloon 5 to the shaft element 1, it is possible to achieve a reliable sealing action in the distal segment 5a protruding beyond the distal end of the shaft element 1 of the kind required, for example, for colorectal irrigation, such as before a colonoscopy. In the closure system according to FIG. 5, with the shaft element lumen 1a extending all the way through and with openings at the distal end and proximal end of the shaft element 1, an additional lumen 100 embodied in the form of an inflation volume inside the wall of the shaft element 1 is provided for the inflation of the intrarectal balloon 5 and the buttressing balloon 6. The lumen 100 in turn communicates via perforations 7 and 8 with the intrarectal balloon 5 and the buttressing balloon 6. The inflation line 4 is introduced into the lumen 100 via the insertion opening 101. The proximal end of the inflation line 4 can be equipped with a closure valve with a pilot balloon 40.

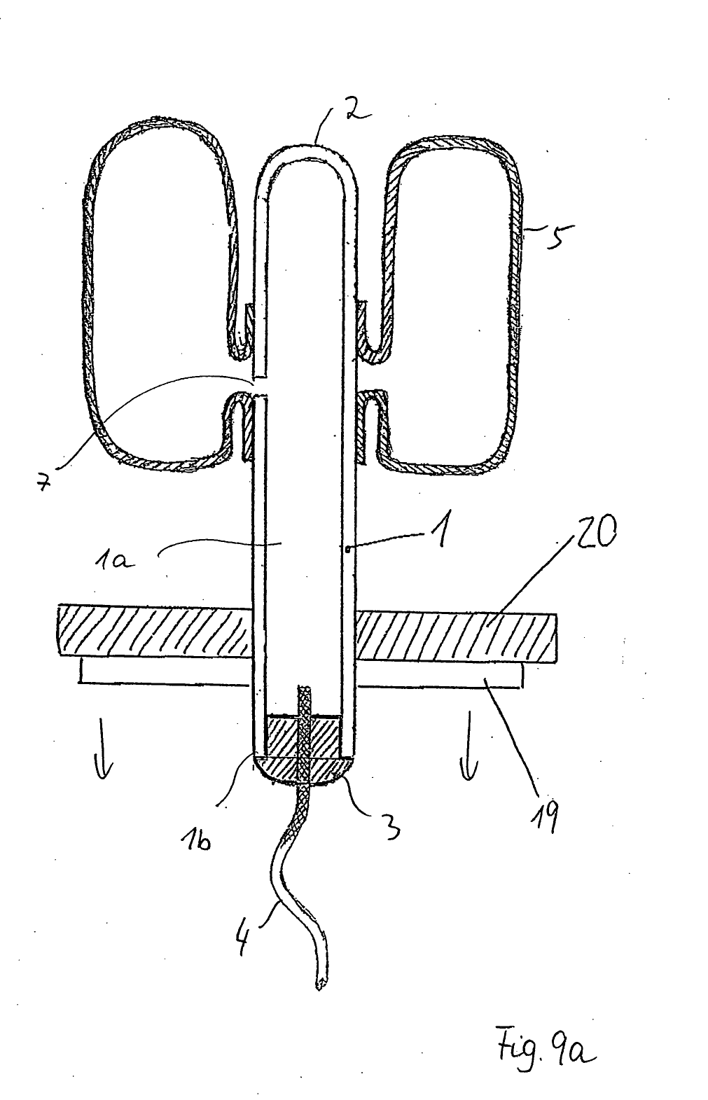

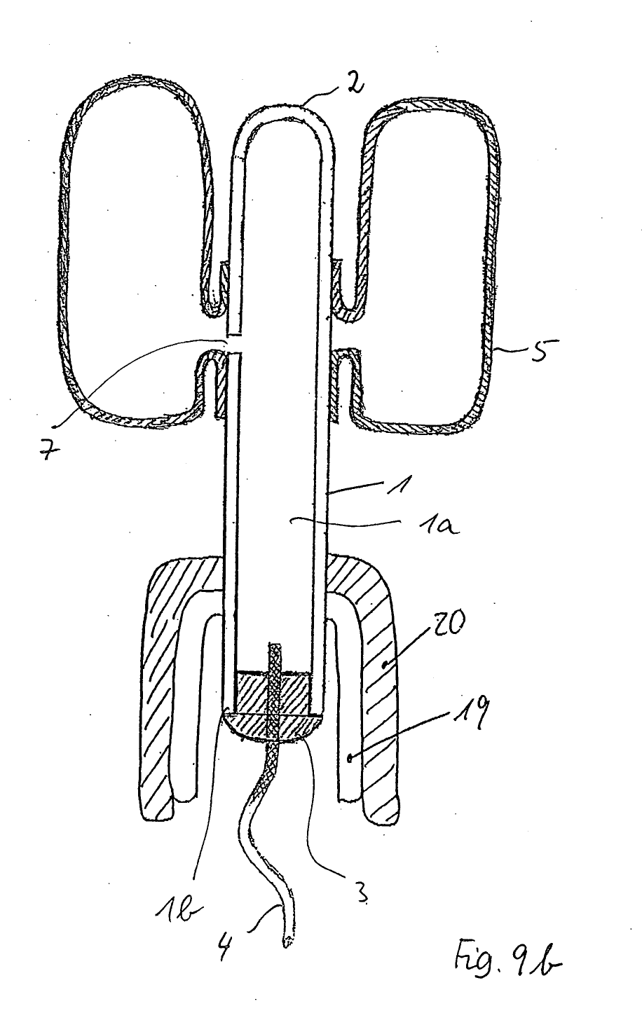

In summary, the closure system according to FIG. 5 performs two sealing functions, namely the low-irritation, axially directed seal acting in the direction of the anus, and the low-resistance, easily sliding seal of an inserted probe, the two sealing functions being performed by one balloon sealing element, namely the intrarectal balloon 5. The anchoring element can be embodied as or in the form of the buttressing balloon 6, according to this invention, but can also be variably embodied, as described, for example, in conjunction with the subsequent FIGS. 8a through 8d, 9a, and 9b.

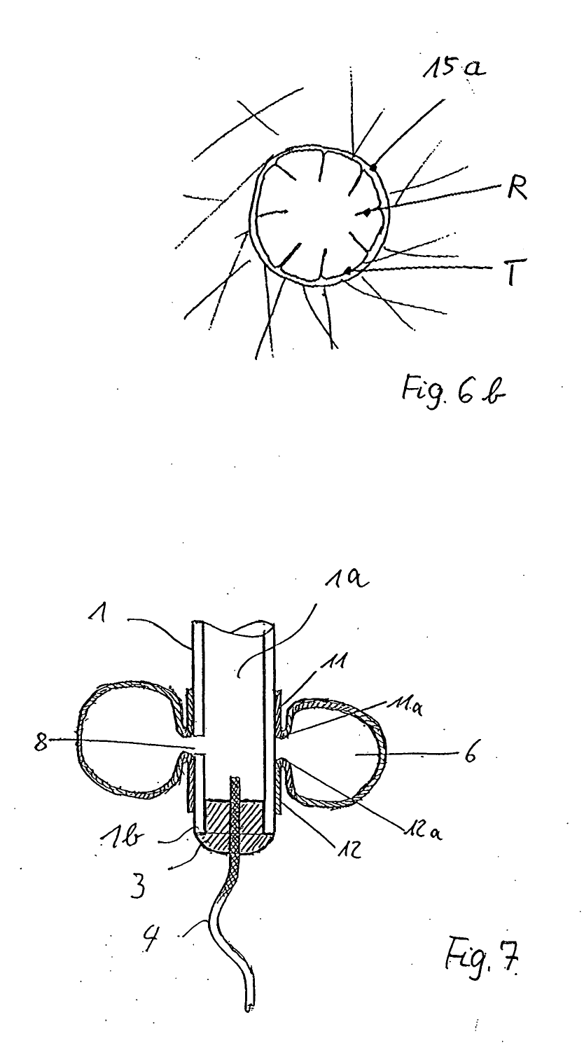

FIG. 6a illustrates an embodiment form of a closure system according to this invention, which is particularly intended for ambulatory and pre-ambulatory treatment of patients with rectal/hemorrhoidal bleeding. The sealing intrarectal balloon 5 in this case is shaped and proportioned so that it extends through the anal canal 15a and transitions in front of the anus into the buttressing balloon 6, which provides the buttressing support, for example comprises one continuous component. To the right of the axial longitudinal axis X of the shaft element 1, FIG. 6a depicts the combined balloon body attached to the shaft element 1 in the inflated, unexpanded or unpressurized, state UZ. On the left side of the axial longitudinal axis X of the shaft element 1, the combined balloon body is shown in the state BZ in which it is inserted into the rectum and pressurized with inflation pressure. In this case, the intrarectal balloon 5 and the buttressing balloon 6 serving as an anchoring and buttressing element comprise a combined extruded tube segment with an initial wall thickness of approx. 80 to 140 μm made of a flexible, extensible plastic material such as polyurethane produced by blowing and shaping in a mold using the blow molding method. The two outer tube ends 10 and 12 are used to attach the combined balloon to the shaft element 1. The intrarectal balloon 5 is again preformed in an essentially cylindrical shape and the buttressing balloon 6 is disk-shaped, the wall thickness is approx. 5 to 45 μm, the tube ends at either end are thicker and have a wall thickness of approx. 20 to 70 μm. Between the two balloon regions 5 and 6, a narrowed section T is blown and molded out of the original tube segment, with a diameter DT smaller than the diameter D of the intrarectal balloon 5 and smaller than the diameter D6 of the buttressing balloon 6. The narrowed section forms the transanal segment T and its dimensions are adapted to the region in which it is used. In a preferred dimensioning in which the intrarectal balloon has a diameter D of 3 to 6 cm and the buttressing balloon 6 has a diameter of 3 to 6 cm or even a diameter slightly smaller than that of the intrarectal balloon but greater than the diameter DT of the narrowed section T, the diameter DT of the narrowed section T should be selected from within a range of 2 to 4 cm, with a length LT of the narrowed section T of 2 to 4 cm.

By contrast, with other embodiments of this invention in which a dilation of the anus is prevented as much as possible, in this case, the diameter of the transanal segment T of the balloon body is significantly larger and is not connected to the shaft element 1 in order to permit a haemostatic compression to be applied to the anal canal and to bleeding venous vessels therein through a slight dilatation of the anal canal. The diameter of the transanal segment T is preferably dimensioned so that it significantly exceeds the diameter of the anus achieved by dilatation. It should measure at least 2 cm.

In the pressurized state according to FIG. 6a, the reserve folds R of the balloon form in the region of the transanal segment T, as shown in the schematically depicted transverse cross-section according to FIG. 6b. The invaginating folding R of the residual balloon sleeve of the transanal segment T in the anal canal 15a makes it possible to set the transmural pressure acting on the tissue equal to the barometrically measured pressure in the balloon, principle of residual diameter, low pressure. As long as the reserve folds R form in the transanal segment T when the balloon is locked in position, then an expansion of the wall of the balloon sleeve in this balloon segment is avoided, which normally requires superelevated, potentially traumatizing inflation pressures that are not required for treatment of bleeding. The folding of the transanal segment T thus makes it possible to precisely control the exposure to force, even in extremely low pressure ranges that are tolerable to the patient, in particular in a range from 10 to 20 mbar. Bleeding that is localized in the immediate vicinity of the anal canal in the terminal rectum portion V is compressed by the sealing pressure, according to this invention, that is exerted on this region in the longitudinal extension of the shaft element. For this type of application, the distal end 2 of the shaft element 1 has the outlet opening 2a through which secretions, blood, or gas can escape. For drainage into a catch receptacle, the closure system according to FIG. 6a can have a corresponding connector K at the proximal end of the shaft element for connection to a drainage tube element. The combined balloon 5, T, 6 is attached to the shaft element 1 using a principle according to this invention, such as shown in FIG. 3 and explained in this specification. Because the uninterrupted lumen 1a of the shaft element 1 serves to convey secretions, blood, or digestive gas, an additional inflation lumen 100 for inflation of the balloon is incorporated into the wall of the catheter shaft and communicates via at least one perforation 7, 8 with the interior of the combined balloon. The filling medium is supplied via the inflation line 4 connected to the lumen 100 by the insertion opening 101.

The above-described equivalence of the barometrically measured inflation pressure and transmural force acting on the balloon in the segment T makes the embodiment of the closure system described ideal for use as a training/biofeedback device. By simply connecting a manometer to the inflation line 4, the sphincter function can be measured in a qualitative and precisely quantitative manner and, through direct feedback to the patient, can be correspondingly trained over time.

In lieu of the above-described use of the closure element shown in FIG. 6a for treatment of patients with rectal/hemorrhoidal bleeding, it is possible to use the closure element according to FIG. 6a for muscular training of the anal muscle. To this end, the transanal segment T is acted on with a presettable pressure, which can be adjusted by the patient, such that a muscular stress is generated that triggers a contraction stimulus of the anus muscle. Connecting a manometer to the pilot balloon 40 thus enables biofeedback training for the patient, for example in order to train the sphincter muscle and regain rectal continence.

In lieu of the anchoring element in the form of an inflatable buttressing balloon according to FIG. 7, it is also possible to use other anchoring elements with a buttressing body as the rectal closure system according to this invention, in connection with a shaft element 1 and an intrarectal balloon 5. FIG. 8a shows a buttress made of foam, in the shape of a conical element 17. FIG. 8b shows a buttressing body made of foam in the shape of a ball 16. FIG. 8c shows a wedge-shaped buttressing body 18 made of foam, which is attached at right angles to the shaft element and can be placed in the anal cleft. FIG. 8d shows another wedge-shaped buttressing body 18.

FIGS. 9a and 9b show one embodiment of a closure system according to this invention with a buttress in the form of an anchoring element that comprises two disk-shaped elements 19, 20, each having a central opening for the passage of the shaft element 1. In this case, the terminal end element 19 of the buttress can comprise a soft, flexible, somatically tolerable plastic such as silicon, polyurethane, or soft PVC, which is permanently attached to the shaft element 1 and can be bent over toward the proximal end 1b of the shaft element 1, such as shown in FIG. 9b, thus permitting it to be placed in the anal cleft in a wedge-like configuration. On the distal side of the terminal end element 19, there is an absorbent, flat layer 20, for example made of a textile nonwoven or felt, that rests directly in front of the anus. When the closure system is reused after being cleaned, this layer element 20 can be replaced.

In lieu of a buttress being attached to the closure system itself, as described in connection with FIGS. 7 through 9, it is also possible for the anchor function to be integrated into a corresponding piece of clothing that assures the buttressing function, such as the patient's underwear. It is thus possible for the closure system to be anchored, for example, by a button at the end, which is inserted into a buttonhole situated in front of the anus in a correspondingly designed pair of underwear. It is also possible to provide pocket-like sew-on patches in a pair of underwear to accommodate the two legs of a support element situated in a T configuration at the end of the shaft element.

According to another embodiment of the closure system of this invention, in one design of the rectal closure, for example as shown in FIG. 1, the shaft element 1 is opened at the distal end 2 by a perforation. It is then possible for digestive gas to escape to the outside through this perforation via the shaft element and through an additional opening provided in the closure cap 3. For odor absorption, the central lumen 1a of the shaft element can be filled with activated charcoal or another filter material. In order to prevent the penetration of digestive secretions into the filter and into the shaft the lumen, the corresponding opening at the distal and 2 of the shaft element can have a patch or membrane made of a material that is gas-permeable, but not fluid-permeable, for example Gore-Tex®. The inflation volume for the balloons 5 and 6 of the closure system according to FIG. 1 must then be embodied inside the wall of the shaft element in addition to the central lumen 1a and must be connected to the balloons 5 and 6 via corresponding perforations.

Because the transanal placement of a large-lumen shaft element is found by many patients to be uncomfortable and can lead to permanent dilation of the anal ring, according to this invention, the segment of the shaft element situated between the intrarectal part of the closure system and the part situated in front of the anus is narrowed or tapered in diameter to such a degree that the shaft element in the anal canal does not cause irritation and is not accompanied by the problems typical of a permanently dilated anal ring, such as a lasting loss of sphincter function.

According to this invention, a balloon-based closure system is used to produce a seal in the event of insufficient anal closure. The seal is produced by a countermovement action of the intrarectal balloon toward the anus largely acting on the anus in the axial direction, which assures a sufficient organ seal even with an extremely low inflation pressure and avoids the irritations and lesions of the terminal rectum that are typical for conventional rectal balloon seals.

German Patent Reference 10 2004 033 425.0, the priority document corresponding to this invention, and its teachings are incorporated, by reference, into this specification.

What is claimed is:

1. A closure system for managing rectal or anal incontinence, having a shaft element with a distal end, a proximal end and at least one lumen, an inflatable intrarectal balloon comprising: a flexible, extensible plastic material with a second distal end and a second proximal end each attached to the shaft element in a securely bonded, sealed fashion, the intrarectal balloon communicating with the at least one lumen via a perforation in a wall of the shaft element and fillable with a medium via the lumen, and an anchoring element acting as a buttress attached to the shaft element and spaced apart from the intrarectal balloon on a proximal side in a direction toward the proximal end of the shaft element, the closure system comprising the second distal end of the intrarectal balloon attached to the shaft element, the balloon being invaginated in a second direction toward the second proximal end of the intrarectal balloon so that the intrarectal balloon has two ends attached to the shaft element, and as the intrarectal balloon is filled with the medium there is a rolling motion from the distal end of the shaft element toward the proximal end of the shaft element.

2. The closure system of claim 1, wherein the second distal end of the intrarectal balloon attached to the shaft element is positioned close to the proximal end of the shaft element at a distance (E) that corresponds to 70% to 20% of the length (L) of the intrarectal balloon in an inflated unexpanded rest state.

3. The closure system of claim 2, wherein the shaft element is of a soft plastic material and has a self-supporting, unbending form with an outer diameter less than or equal to 13 mm.

4. The closure system of claim 3, wherein the shaft element has a closed atraumatically designed distal end and a central lumen that extends from the open proximal end to the distal end, and the central lumen can be closed at the open proximal end by a removable closure element that contains an inflation line for supplying the medium through the lumen.

5. The closure system of claim 3, wherein the shaft element has the distal end open and a lumen that extends from the proximal end to the distal end, inside the wall of the shaft element there is an additional lumen for supplying the medium which is connectible to an inflation line via an opening in a proximal region of the shaft element, and through at least one perforation in the wall of the shaft element communicates with an interior of the intrarectal balloon to be inflated.

6. The closure system of claim 5, wherein the inflation line has an automatic closing valve for the medium, which has a pilot balloon for gaging a respective inflation pressure.

7. The closure system of claim 6, wherein the intrarectal balloon comprises an extruded tube segment with a wall thickness in a range from 80 to 140 μm and made of a flexible, extensible plastic material blown into one of an approximate cylinder shape and an approximate roller shape with a blow molding method to reduce a wall thickness of the balloon to 5 to 25 μm, two tube ends remaining at the two ends of the balloon are blown to a second wall thickness of approximately 40 μm to 60 μm for insertion and attachment of the shaft element.

8. The closure system of claim 7, wherein the intrarectal balloon is made of a polyurethane.

9. The closure system of claim 8, wherein in the inflated unexpanded rest state, the intrarectal balloon has a diameter (D) of approximately 3 to 6 cm and a length (L) of approximately 5 to 10 cm.

10. The closure system of claim 9, wherein the second proximal end of the intrarectal balloon is attached to the shaft element and spaced 2 to 4 cm apart from the anchoring element.

11. The closure system of claim 10, wherein a cylindrically shaped region of the intrarectal balloon has a wall thickness of 5 to 15 μm.

12. The closure system of claim 11, wherein an inflation pressure of the intrarectal balloon is 10 to 30 mbar.

13. The closure system of claim 11, wherein an inflation pressure of the intrarectal balloon is 10 to 20 mbar.

14. The closure system of claim 13, wherein the inflated intrarectal balloon exerts an axial pressure oriented toward the proximal end of the shaft element, and the axial pressure is adjustable by the inflation pressure.

15. The closure system of claim 14, wherein the perforation in the wall of the shaft element which connects the lumen that supplies the medium to the inside of the intrarectal balloon is close to the second proximal tube end of the intrarectal balloon attached to the shaft element.

16. The closure system of claim 15, wherein one of a disk-shaped and a spherical shaped buttressing balloon that comprises a flexible extensible plastic material acts as an anchoring element that can be acted on by and inflated with the medium.

17. The closure system of claim 16, wherein the buttressing balloon comprises an extruded tube segment made of a flexible extensible plastic material with a wall thickness of approximately 80 to 200 μm in a disk-shaped cylindrical form by reducing the wall thickness to 5 to 45 μm in the blow molding process, and the two ends of the buttressing balloon are blown to have a second wall thickness of approximately 40 to 80 μm for insertion and attachment of the shaft element.

18. The closure system claim 17, wherein the buttressing balloon is made of a polyurethane.

19. The closure system of claim 18, wherein the buttressing balloon and the intrarectal balloon communicate with the same lumen of the shaft element for supplying the medium.

20. The closure system of claim 19, wherein the second proximal end and the second distal tube end of the buttressing balloon are attached to the shaft element close to the perforation in the wall of the shaft element which connects the lumen supplying the medium to the interior of the buttressing balloon.

21. The closure system of claim 20, wherein the intrarectal balloon and the buttressing balloon comprise a shared extruded tube segment of a flexible extensible plastic material with a third distal end and a third proximal end by being blown and formed in a mold, between the third distal end and the third proximal end a tubular connecting piece provides spacing between the two balloon formations for the intrarectal balloon and the buttressing balloon, and the connecting piece has a diameter smaller than that of the intrarectal balloon and the third distal end and the third proximal end of the tube segment and the tubular connecting piece are connected to the shaft element.

22. The closure system of claim 15, wherein the anchoring element has a buttressing body of a soft elastic foam.

23. The closure system of claim 22, wherein in a direction of the longitudinal section through the shaft element the buttressing body has a shape of one of a ball, of a roller, a sphere, a cone and a wedge.

24. The closure system of claim 15, wherein the anchoring element is a disk-shaped flexible plate that comprises a soft somatically tolerable plastic material permanently attached to the shaft element, which can be bent over toward the proximal end of the shaft element.

25. The closure system of claim 24, wherein an absorbent flat layer is mountable in a replaceable manner on a fourth distal side of the plate acting as the anchoring element.

26. The closure system of claim 5, wherein a closure body positioned at the proximal end of the shaft element has an opening to permit digestive gas to escape to an outside through the lumen from the opening at the distal end, and the opening at the distal end of the shaft element is covered with a membrane comprising a gas-permeable and non-fluid-permeable material.

27. The closure system of claim 26, wherein the lumen extending from the distal end to the proximal end of the shaft element contains a filter material for odor absorption.

28. The closure system of claim 26, wherein the distal, open end of the shaft element ends at attachment regions of the second distal end of the intrarectal balloon and a probe can be introduced through the central lumen extending all the way through the shaft element.

29. The closure system of claim 20, wherein the intrarectal balloon and the buttressing balloon comprise an extruded tube segment of a flexible extensible plastic material made by being blown and shaped in a mold, and between the intrarectal balloon and the buttressing balloon a narrowed section has a diameter smaller than a second diameter of the intrarectal balloon and the buttressing balloon.

30. The closure system of claim 29, wherein a diameter of the intrarectal balloon is 3 to 6 cm, a second diameter of the buttressing balloon is 3 to 6 cm, a third diameter of the narrowed segment is 2 to 4 cm, and a length of the narrowed section is 2 to 4 cm, with respect to the inflated intrarectal balloon in the unexpanded rest state.

31. The closure system of claim 1, wherein the shaft element is of a soft plastic material and has a self-supporting, unbending form with an outer diameter less than or equal to 13 mm.

32. The closure system of claim 1, wherein the shaft element has a closed atraumatically designed distal end and a central lumen that extends from the open proximal end to the distal end, and the central lumen can be closed at the open proximal end by a removable closure element that contains an inflation line for supplying the medium through the lumen.

33. The closure system of claim 1, wherein the shaft element has the distal end open and a lumen that extends from the proximal end to the distal end, inside the wall of the shaft element there is an additional lumen for supplying the medium which is connectible to an inflation line via an opening in a proximal region of the shaft element, and through at least one perforation in the wall of the shaft element communicates with an interior of the intrarectal balloon to be inflated.

34. The closure system of claim 1, wherein the inflation line has an automatic closing valve for the medium, which has a pilot balloon for gaging a respective inflation pressure.

35. The closure system of claim 1, wherein the intrarectal balloon comprises an extruded tube segment with a wall thickness in a range from 80 to 140 μm and made of a flexible, extensible plastic material blown into one of an approximate cylinder shape and an approximate roller shape with a blow molding method to reduce a wall thickness of the balloon to 5 to 25 μm, two tube ends remaining at the two ends of the balloon are blown to a second wall thickness of approximately 40 μm to 60 μm for insertion and attachment of the shaft element.

36. The closure system of claim 1, wherein the intrarectal balloon is made of a polyurethane.

37. The closure system of claim 1, wherein in the inflated unexpanded rest state, the intrarectal balloon has a diameter (D) of approximately 3 to 6 cm and a length (L) of approximately 5 to 10 cm.

38. The closure system of claim 1, wherein the second proximal end of the intrarectal balloon is attached to the shaft element and spaced 2 to 4 cm apart from the anchoring element.

39. The closure system of claim 1, wherein a cylindrically shaped region of the intrarectal balloon has a wall thickness of 5 to 15 μm.

40. The closure system of claim 1, wherein an inflation pressure of the intrarectal balloon is 10 to 30 mbar.

41. The closure system of claim 1, wherein an inflation pressure of the intrarectal balloon is 10 to 20 mbar.

42. The closure system of claim 1, wherein the inflated intrarectal balloon exerts an axial pressure oriented toward the proximal end of the shaft element, and the axial pressure is adjustable by the inflation pressure.

43. The closure system of claim 1, wherein the perforation in the wall of the shaft element which connects the lumen that supplies the medium to the inside of the intrarectal balloon is close to the second proximal tube end of the intrarectal balloon attached to the shaft element.

44. The closure system of claim 1, wherein one of a disk-shaped and a spherical shaped buttressing balloon that comprises a flexible extensible plastic material acts as an anchoring element that can be acted on by and inflated with the medium.

45. The closure system of claim 44, wherein the buttressing balloon comprises an extruded tube segment made of a flexible extensible plastic material with a wall thickness of approximately 80 to 200 μm in a disk-shaped cylindrical form by reducing the wall thickness to 5 to 45 μm in the blow molding process, and the two ends of the buttressing balloon are blown to have a second wall thickness of approximately 40 to 80 μm for insertion and attachment of the shaft element.

46. The closure system claim 10, wherein the buttressing balloon is made of a polyurethane.

47. The closure system of claim 16, wherein the buttressing balloon and the intrarectal balloon communicate with the same lumen of the shaft element for supplying the medium.

48. The closure system of claim 16, wherein the second proximal end and the second distal tube end of the buttressing balloon are attached to the shaft element close to the perforation in the wall of the shaft element which connects the lumen supplying the medium to the interior of the buttressing balloon.

49. The closure system of claim 1, wherein the intrarectal balloon and the buttressing balloon comprise a shared extruded tube segment of a flexible extensible plastic material with a third distal end and a third proximal end by being blown and formed in a mold, between the third distal end and the third proximal end a tubular connecting piece provides spacing between the two balloon formations for the intrarectal balloon and the buttressing balloon, and the connecting piece has a diameter smaller than that of the intrarectal balloon and the third distal end and the third proximal end of the tube segment and the tubular connecting piece are connected to the shaft element.

50. The closure system of claim 1, wherein the anchoring element has a buttressing body of a soft elastic foam.

51. The closure system of claim 50, wherein in a direction of the longitudinal section through the shaft element the buttressing body has a shape of one of a ball, of a roller, a sphere, a cone and a wedge.

52. The closure system of claim 1, wherein the anchoring element is a disk-shaped flexible plate that comprises a soft somatically tolerable plastic material permanently attached to the shaft element, which can be bent over toward the proximal end of the shaft element.

53. The closure system of claim 52, wherein an absorbent flat layer is mountable in a replaceable manner on a fourth distal side of the plate acting as the anchoring element.

54. The closure system of claim 33, wherein a closure body positioned at the proximal end of the shaft element has an opening to permit digestive gas to escape to an outside through the lumen from the opening at the distal end, and the opening at the distal end of the shaft element is covered with a membrane comprising a gas-permeable and non-fluid-permeable material.

55. The closure system of claim 54, wherein the lumen extending from the distal end to the proximal end of the shaft element contains a filter material for odor absorption.

56. The closure system of claim 5, wherein the distal, open end of the shaft element ends at attachment regions of the second distal end of the intrarectal balloon and a probe can be introduced through the central lumen extending all the way through the shaft element.

57. The closure system of claim 1, wherein the intrarectal balloon and the buttressing balloon comprise an extruded tube segment of a flexible extensible plastic material made by being blown and shaped in a mold, and between the intrarectal balloon and the buttressing balloon a narrowed section has a diameter smaller than a second diameter of the intrarectal balloon and the buttressing balloon.

58. The closure system of claim 57, wherein a diameter of the intrarectal balloon is 3 to 6 cm, a second diameter of the buttressing balloon is 3 to 6 cm, a third diameter of the narrowed segment is 2 to 4 cm, and a length of the narrowed section is 2 to 4 cm, with respect to the inflated intrarectal balloon in the unexpanded rest state.
